(19) United States
(12) Patent Application Publication
(10) Pub. No.: US 2007/0122358 A1
(43) Pub. Date: May 31, 2007

(54) Dentifrice composition free of abrasive material

(75) Inventors: Xiaoli Wang, Beijing (CN); Lijiang Yang, Beijing (CN); Yun Wang, Beijing (CN); Ying Cheng, Beijing (CN)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/605,563

(22) Filed: Nov. 28, 2006

Related U.S. Application Data
(60) Provisional application No. 60/740,538, filed on Nov. 29, 2005.
(60) Provisional application No. 60/751,405, filed on Dec. 16, 2005.

(51) Int. Cl. A61K 8/21; A61K 8/46

(52) U.S. Cl. 424/52; 424/57

(57) ABSTRACT

A dentifrice composition comprising, (a) a hydrophilic clay material, (b) a phytic acid compound, (c) an effective amount of an oral care active, and (d) a polar solvent carrier; wherein the composition is substantially free of abrasive material.

### CROSS REFERENCE TO RELATED APPLICATION(S)

This application claims the benefit of U.S. Provisional Application No. 60/740,538, filed on Nov. 29, 2005; and U.S. Provisional Application No. 60/751,405 filed on Dec. 16, 2005.

### FIELD OF THE INVENTION

The present invention relates to a dentifrice composition which is substantially free of abrasive material, and which can still maintain the cleaning benefits of a regular toothpaste.

### BACKGROUND

Dentifrice compositions such as toothpastes are routinely used by consumers as part of their oral care hygiene regimens. It is well known that oral care products can provide both therapeutic and cosmetic hygiene benefits to consumers. Therapeutic benefits include, but are not limited to, caries prevention, gingivitis prevention, and hypersensitivity control. Cosmetic benefits include, but are not limited to, control of plaque and calculus formulation, removal and prevention of tooth stain, tooth whitening, breath freshening, and mouth feel aesthetics such as fresh sensation or slippery sensory feel.

Usage of abrasive material such as silica has long been considered critical for delivering the above mentioned therapeutic and cosmetic benefits of a dentifrice composition. Abrasive materials provide physical abrasion between toothbrush and teeth to clean plaque, stain, and calculus, while also build rheology and structure of the dentifrice for maintaining thermal stability of the overall formulation.

At the same time, however, usage of abrasive materials may also have certain negative effects to overall formulation. For example, the cost of silica itself is expensive. Further, silica adsorbs components in a typical dentifrice composition such as active agents, flavors, and foaming agents, thereby the concentrations of these components must be adjusted according to the foreseen loss by silica. Still further, certain active agents such as cetylpyridinium chloride have poor compatibility with silica, and therefore cannot be included in a dentifrice composition, despite its known effectiveness. In another aspect, it is a conception of the general consumer that abrasive materials may erode the enamel, if the teeth are brushed too intensively.

Based on the foregoing, there is a need for a dentifrice composition free of abrasives, which can still deliver the benefits of a regular dentifrice composition. Specifically, there is a need for an abrasive-free dentifrice composition which is thermally stable, and which can deliver the same cleaning benefits as abrasive-including compositions on the market.

### SUMMARY

The present invention is directed to a dentifrice composition comprising:

(a) a hydrophilic clay material;

(b) a phytic acid compound;

(c) an effective amount of an oral care active; and

(d) a polar solvent carrier;

wherein the composition is substantially free of abrasive material.

The present invention is further directed to a method of cleaning the teeth absent an abrasive.

These and other features, aspects, and advantages of the present invention will become evident to those skilled in the art from a reading of the present disclosure with the appended claims.

### DETAILED DESCRIPTION

The following is a list of definitions for terms used herein.

“Comprising” means that other steps and other ingredients which do not affect the end result can be added. This term encompasses the terms “consisting of” and “consisting essentially of”.

All percentages are by weight of total composition unless specifically stated otherwise.

All cited references are incorporated herein by reference in their entireties. Citation of any reference is not an admission regarding any determination as to its availability as prior art to the claimed invention.

All ratios are weight ratios unless specifically stated otherwise.

The present invention, in its product and process aspects, is described in detail as follows.

a) Hydrophilic Clay Material

The composition of the present invention comprises a hydrophilic clay material which swells or thickens with the presence of a polar solvent, and acts as a binder for the dentifrice composition. The hydrophilic clay material may be any known that is safe and aesthetically acceptable for oral care use. The hydrophilic clay material provides a suitable rheology for the present dentifrice composition of various product form, including brushing liquids, gels, and toothpaste, having a viscosity of from about 1 mPas to about 450,000 Pas as measured by Brookfield viscometer with T-E spindle. In one highly preferred embodiment, the present dentifrice composition is a gel or toothpaste of suitable body and viscosity, the viscosity being from about 10,000 Pas to about 450,000 Pas, preferably from about 10,000 Pas to about 250,000 Pas. Additional binders may be added to provide such suitable rheology, as later discussed.

It has been surprisingly found that, hydrophilic clay material provides the above mentioned suitable stability at relatively low level, and relatively low cost, without the existence of an abrasive material in the composition.

The hydrophilic clay material is preferably comprised, by weight of the entire composition, at from about 0.01% to about 10%, preferably from about 1% to about 5%.

Hydrophilic clay materials useful herein include natural and synthesized layered silicate minerals, fumed silicas, thickening precipitated silica, and mixtures thereof. Layered silicate minerals may be naturally occurred, or synthesized to have magnesium substituted with certain portions of the mineral. The fumed silicas are those that provide very little to no abrasive function having a particle size of smaller than about 5 μm, typically from about 1 nm to about 1 μm. The thickening precipitated silica are those having a DOA value of at least about 150 ml/100 g, preferably at least about 250 ml/100 g, and having a particle size of about 1 μm to about 50 μm. The thickening precipitated silica can be distinguished from abrasive silica material, due to its high oil absorbing capability, as defined by the DOA value. Such capability provides thickening property.

Commercially available hydrophilic clay materials useful herein include synthesized layered magnesium silicate by the tradename LAPONITE series available from Rockwood Additives Limited and Southern Clay Company, fumed silica having an average particle size of about 12 nm by the tradename AEROSOL series and CAB-O-SIL available from Cabot & Degussa Corporation, amorphous precipitated silica by the tradename ZEODENT 165 from J. M. Huber Company and SYLOX 15 from Grace Davision, and precipitated silica by the tradename TIXOSIL from Rhodia.

b) Phytic Acid Compound

The composition of the present invention comprises a phytic acid compound in an amount it provides effective cleaning to the teeth. Phytic acid, also known as myo-inositol hexaphosphate, or inositol hexaphosphoric acid, is a natural vegetable based biodegradable chelating agent in liquid form with chelating performance comparable to that of EDTA. Origin of vegetables from which phytic acid compounds can be found, include cereal grains, legumes, nuts oilseeds, pollen, spore, and organic soils.

The phytic acid compound herein can be phytic acid itself and/or its orally acceptable salts including, but not limited to, alkali metal salts and alkali earth metal salts. Useful phytic acid salts include sodium phytate, potassium phytate, magnesium phytate, calcium phytate, stannous phytate, zinc phytate, copper phytate, ferrum phytate, and mixtures thereof.

It has been surprisingly found that the phytic acid compound provides good cleaning effect in the present abrasive free composition without consumer noticeable staining, and is compatible with the present polar solvent carrier, as well as a wide variety of oral care active ingredients. Without being bound by theory, it is believed that the phytic acid compound provides unique good cleaning due to its liquid form, by flowing between teeth. Such flowing is believed to provide cleaning in a way differently from solid abrasive materials. Further, it is believed that the phytic acid compound provides a positive effect for protecting tooth enamel from acid dissolution, and anti-tartar effect.

Commercially available phytic acid compounds useful herein include phytic acid solution, sodium phytate, magnesium phytate, calcium phytate, and stannous phytate available from Sichuan Chengdu Yason, Shikishima Starch Manufacturing Company, and Nibbio.

c) Oral Care Active

The composition of the present invention comprises an oral care active which delivers the intended therapeutic oral care benefit. Oral care actives useful herein include anti-calculus agents, stannous ion sources, fluoride ion sources, whitening agents, anti-microbial agents, anti-plaque agents, anti-inflammatory agents, nutrients, antioxidants, anti-viral agents, analgesic and anesthetic agents, zinc-containing layered material, and mixtures thereof.

c-1) Anticalculus Agent

Useful oral care agents herein include an anti-calculus agent, which in one embodiment may be present from about 0.05% to about 50%, by weight of the oral care composition, in another embodiment is from about 0.05% to about 25%. The anti-calculus agent may be selected from the group consisting of polyphosphates (including pyrophosphates) and salts thereof; polyamino propane sulfonic acid (AMPS) and salts thereof; polyolefin sulfonates and salts thereof; polyvinyl phosphates and salts thereof; polyolefin phosphates and salts thereof; diphosphonates and salts thereof; phosphonoalkane carboxylic acid and salts thereof; polyphosphonates and salts thereof; polyvinyl phosphonates and salts thereof; polyolefin phosphonates and salts thereof; polypeptides; and mixtures thereof. In one embodiment, the salts are alkali metal salts. Polyphosphates are generally employed as their wholly or partially neutralized water-soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation, which are commercially known as Sodaphos (n≈6), Hexaphos (n≈13), and Glass H (n≈21, sodium hexametaphosphate), and mixtures thereof. The pyrophosphate salts useful in the present invention include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophosphate, tetrasodium pyrophosphate (Na₄P₂O₇), tetrapotassium pyrophosphate (K₄P₂O₇), and mixtures thereof. Polyolefin sulfonates include those wherein the olefin group contains 2 or more carbon atoms, and salts thereof. Polyolefin phosphonates include those wherein the olefin group contains 2 or more carbon atoms. Polyvinylphosphonates include polyvinylphosphonic acid. Diphosphonates and salts thereof include azocycloalkane-2,2-diphosphonic acids and salts thereof, ions of azocycloalkane-2,2-diphosphonic acids and salts thereof, azacyclohexane-2,2-diphosphonic acid, azacyclopentane-2,2-diphosphonic acid, N-methyl-azacyclopentane-2,3-diphosphonic acid, EHDP (ethane-1-hydroxy-1,1,-diphosphonic acid), AHP (azacycloheptane-2,2-diphosphonic acid), ethane-1-amino-1,1-diphosphonate, dichloromethane-diphosphonate, etc. Phosphonoalkane carboxylic acid or their alkali metal salts include PPTA (phosphonopropane tricarboxylic acid), PBTA (phosphonobutane-1,2,4-tricarboxylic acid), each as acid or alkali metal salts. Polyolefin phosphates include those wherein the olefin group contains 2 or more carbon atoms. Other useful material include synthetic anionic polymers, including polyacrylates and copolymers of maleic anhydride or acid and methyl vinyl ether (e.g., Gantrez), as described, for example, in U.S. Pat. No. 4,627,977, as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof. Additionally, the oral care composition can include a polymer carrier, such as those described in U.S. Pat. Nos. 6,682,722 and 6,589,512 and U.S. application Ser. Nos. 10/424,640 and 10/430,617.

c-2) Stannous Ion Source

Useful oral care agents herein include a stannous ion source. The stannous ions may be provided from stannous fluoride and/or other stannous salts. Stannous fluoride has been found to help in the reduction of gingivitis, plaque, sensitivity, and in improved breath benefits. The stannous ions provided in an oral composition will provide efficacy to a subject using the composition. Although efficacy could include benefits other than the reduction in gingivitis, efficacy is defined as a noticeable amount of reduction in in situ plaque metabolism. Formulations providing such efficacy typically include stannous levels provided by stannous fluoride and/or other stannous salts ranging from about 3,000 ppm to about 15,000 ppm stannous ions in the total composition. Below about 3,000 ppm stannous the efficacy of the stannous is not sufficient. The stannous ion is present in an amount of from about 4,000 ppm to about 12,000 ppm, in one embodiment from about 5,000 ppm to about 10,000 ppm. Other stannous salts include organic stannous carboxylates, such as stannous acetate, stannous gluconate, stannous oxalate, stannous malonate, stannous citrate, stannous ethylene glycoxide, stannous formate, stannous sulfate, stannous lactate, stannous tartrate, and the like. Other stannous ion sources include, stannous halides such as stannous chlorides, stannous bromide, stannous iodide and stannous chloride dihydride. In one embodiment the stannous ion source is stannous fluoride in another embodiment, stannous chloride dihydrate. The combined stannous salts may be present in an amount of from about 0.001% to about 11%, by weight of the compositions. The stannous salts may, in one embodiment, be present in an amount of from about 0.01% to about 7%, in another embodiment from about 0.1% to about 5%, and in another embodiment from about 1.5% to about 3%, by weight of the composition.

c-3) Fluoride Ion Source

Useful oral care agents herein include a fluoride ion source to provide an anticaries effect. Among these materials are inorganic fluoride salts, such as soluble alkali metal fluoride salts, for example, sodium fluoride, potassium fluoride, sodium monofluorophosphate and sodium hexafluorosilicate. Alkali metal fluorides, such as sodium fluoride, sodium monofluorophosphate, sodium hexafluorosilicate and mixtures thereof, are preferred.

The amount of fluorine-providing salt is generally present in the oral composition at a concentration of about 0.0 to about 3.0% by weight. Any suitable minimum amount of such salt may be used, but it is preferable to employ sufficient fluoride salt to release from about 50 ppm to about 3500 ppm, preferably from about 300 ppm to 2,000 ppm, of fluoride ion.

c-4) Whitening Agent

Useful oral care agents herein include a whitening agent The actives suitable for whitening are selected from the group consisting of alkali metal and alkaline earth metal peroxides, metal chlorites, perborates inclusive of mono and tetrahydrates, perphoshates, percarbonates, peroxyacids, alkali metal and persulfates, such as ammonium, potassium, sodium and lithium persulfates, and combinations thereof. Suitable peroxide compounds include hydrogen peroxide, urea peroxide, calcium peroxide, carbamide peroxide, magnesium peroxide, zinc peroxide, strontium peroxide and mixtures thereof. In one embodiment the peroxide compound is carbamide peroxide. Suitable metal chlorites include calcium chlorite, barium chlorite, magnesium chlorite, lithium chlorite, sodium chlorite, and potassium chlorite. Additional whitening actives may be hypochlorite and chlorine dioxide. In one embodiment the chlorite is sodium chlorite. In another embodiment the percarbonate is sodium percarbonate. In one embodiment the persulfates are oxones. The level of these substances is dependent on the available oxygen or chlorine, respectively, that the molecule is capable of providing to bleach the stain. In one embodiment the whitening agents may be present at levels from about 0.01% to about 40%, in another embodiment from about 0.1% to about 20%, in another embodiment form about 0.5% to about 10%, and in another embodiment from about 4% to about 7%, by weight of the composition.

c-5) Anti-Microbial Agent

Useful oral care agents herein include other anti-microbial agents. Such agents may include, but are not limited to: 5-chloro-2-(2,4-dichlorophenoxy)-phenol, commonly referred to as triclosan; 8-hydroxyquinoline and its salts; copper II compounds, including, but not limited to, copper(II) chloride, copper(II) sulfate, copper(II) acetate, copper(II) fluoride and copper(II) hydroxide; phthalic acid and its salts including, but not limited to those disclosed in U.S. Pat. No. 4,994,262, including magnesium monopotassium phthalate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; iodine; sulfonamides; bisbiguanides; phenolics; delmopinol, octapinol, and other piperidino derivatives; niacin preparations; zinc or stannous ion agents such as zinc oxide, zinc lactate and zinc citrate; nystatin; grapefruit extract; apple extract; thyme oil; thymol; antibiotics such as augmentin, amoxicillin, tetracycline, doxycycdine, minocycline, metronidazole, neomycin, kanamycin, cetylpyridinium chloride, and clindamycin; analogs and salts of the above; essential oils including thymol, geraniol, carvacrol, citral, hinokitiol, eucalyptol, catechol (particularly 4-allyl catechol) and mixtures thereof; methyl salicylate; hydrogen peroxide; metal salts of chlorite; and mixtures of all of the above. Anti-microbial components may be present from about 0.001% to about 20% by weight of the composition.

c-6) Anti-Plaque Agent

Useful oral care agents herein include other anti-plaque agent such as dimethyl isosorbide, copper salts, strontium salts, magnesium salts or a dimethicone copolyol. The dimethicone copolyol is selected from C12 to C20 alkyl dimethicone copolyols and mixtures thereof. In one embodiment the dimethicone copolyol is cetyl dimethicone copolyol marketed under the Trade Name Abil EM90. The dimethicone copolyol in one embodiment can be present in a level of from about 0.001% to about 25%, in another embodiment from about 0.01% to about 5%, and in another embodiment from about 0.1% to about 1.5% by weight of the composition.

c-7) Anti-Inflammatory Agent

Useful oral care agents herein include anti-inflammatory agents. Such agents may include, but are not limited to, non-steroidal anti-inflammatory agents oxicams, salicylates, propionic acids, acetic acids and fenamates. Such NSAIDs include but are not limited to ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone and acetaminophen. Use of NSAIDs such as ketorolac are claimed in U.S. Pat. No. 5,626,838. Disclosed therein are methods of preventing and/or treating primary and reoccurring squamous cell carcinoma of the oral cavity or oropharynx by topical administration to the oral cavity or oropharynx of an effective amount of an NSAID. Suitable steroidal anti-inflammatory agents include corticosteroids, such as fluccinolone, and hydrocortisone.

c-8) Nutrients

Useful oral care agents herein include nutrients which improve the condition of the oral cavity. Nutrients include minerals, vitamins, oral nutritional supplements, enteral nutritional supplements, and mixtures thereof. Useful minerals include calcium, phosphorus, zinc, manganese, potassium and mixtures thereof. Vitamins can be included with minerals or used independently. Suitable vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Oral nutritional supplements include amino acids, lipotropics, fish oil, and mixtures thereof. Amino acids include, but are not limited to L-Tryptophan, L-Lysine, Methionine, Threonine, Levocamitine or L-carnitine and mixtures thereof. Lipotropics include, but are not limited to, choline, inositol, betaine, linoleic acid, linolenic acid, and mixtures thereof. Fish oil contains large amounts of Omega-3 (N-3) polyunsaturated fatty acids, eicosapentaenoic acid and docosahexaenoic acid. Enteral nutritional supplements include, but are not limited to, protein products, glucose polymers, corn oil, safflower oil, medium chain triglycerides. Minerals, vitamins, oral nutritional supplements and enteral nutritional supplements are described in more detail in Drug Facts and Comparisons (loose leaf drug information service), Wolters Kluer Company, St. Louis, Mo., ©1997, pps. 3-17 and 54-57.

c-9) Antioxidants

Useful oral care agents herein include antioxidants. Antioxidants are disclosed in texts such as Cadenas and Packer, The Handbook of Antioxidants, © 1996 by Marcel Dekker, Inc. Antioxidants useful in the present invention include, but are not limited to, Vitamin E, ascorbic acid, Uric acid, carotenoids, Vitamin A, flavonoids and polyphenols, herbal antioxidants, melatonin, aminoindoles, lipoic acids and mixtures thereof.

c-10) Anti-Viral Agents

Useful oral care agents herein include an antiviral actives used to treat viral infections. Such antiviral actives include, but are not limited to: phosphonoformic acid; cyosine derivatives; purine analogues, such as adenosine, guanosine and inosine analogues; pyrimidine bases, such as citidine and thymidine; amantadines; rimantadine HCl; ribavirin; zanamivir; oseltamivir phosphate; trifluridine; heterocyclic dyes; acyclovir; famciclovir; valacyclovir, cidofovir; ganciclovir; levimisole; idoxuridine; lipophilic β-ketones; and thiosemicarbazones. These antiviral actives are described in Drug Facts and Comparisons (loose-leaf drug information service), Wolters Kluwer Company, St. Louis, Mo., ©2001, pp. 1400-1423(b), and in Kirk-Othmer, Encyclopedia of Chemical Technology, Fourth Edition, Volume 3, Wiley-Interscience Publishers (1992), pp. 576-607.

c-11) Analgesic and Anesthetic Agents

Useful oral care agents herein include anti-pain or desensitizing agents. Analgesics are agents that relieve pain by acting centrally to elevate pain threshold without disturbing consciousness or altering other sensory modalities. Such agents may include, but are not limited to: strontium chloride; potassium nitrate; sodium fluoride; sodium nitrate; acetanilide; phenacetin; acertophan; thiorphan; spiradoline; aspirin; codeine; thebaine; levorphenol; hydromorphone; oxymorphone; phenazocine; fentanyl; buprenorphine; butaphanol; nalbuphine; pentazocine; natural herbs, such as gall nut; Asarum; Cubebin; Galanga; scutellaria; Liangmianzhen; and Baizhi. Anesthetic agents, or topical analgesics, such as acetaminophen, sodium salicylate, trolamine salicylate, lidocaine and benzocaine may also be present. These analgesic actives are described in detail in Kirk-Othmer, Encyclopedia of Chemical Technology, Fourth Edition, Volume 2, Wiley-Interscience Publishers (1992), pp. 729-737.

c-12) Zinc-Containing Layered Material

Useful as the oral care active herein are zinc-containing layered material which have effective anti-microbial and anti-gingivitis benefit due to high zinc lability. Those zinc-containing layered materials particularly useful herein are those having a relative zinc lability of greater than about 25%, have an average particle size of less than about 20 microns, and have a high surface area. In one preferred embodiment, the zinc-containing layered material herein is incorporated in compositions containing an anionic surfactant, as later discussed.

Zinc lability is a measure of the chemical availability of zinc ion. Soluble zinc salts that do not complex with other species in solution have a relative zinc lability, by definition, of 100%. The use of partially soluble forms of zinc salts and/or incorporation in a matrix with potential complexants generally lowers the zinc lability substantially below the defined 100% maximum. Labile zinc is maintained by choice of an effective zinc-containing layered material or formation of an effective zinc-containing layered material in-situ by known methods.

Zinc lability is assessed by combining a diluted zinc-containing solution or dispersion with the metallochromic dye xylenol orange (XO) and measurement of the degree of color change under specified conditions. The magnitude of color formation is proportional to the level of labile zinc. The procedure developed has been optimized for aqueous surfactant formulations but may be adapted to other physical product forms as well.

A spectrophotometer is used to quantify the color change at 572 nm, the wavelength of optimum color change for XO. The spectrophotometer is set to zero absorbance at 572 nm utilizing a product control as close in composition to the test product except excluding the potentially labile form of zinc. The control and test products are then treated identically as follows. A 50 μl product sample is dispensed into ajar and 95 ml of deaerated, distilled water are added and stirred. 5 mL of a 23 mg/mL xylenol orange stock solution at pH 5.0 is pipetted into the sample jar; this is considered time 0. The pH is then adjusted to 5.50±0.01 using dilute HCl or NaOH. After 10.0 minutes, a portion of the sample is filtered (0.45μ) and the absorbance measured at 572 nm. The measured absorbance is then compared to a separately measured control to determine the relative zinc lability (zero TO 100%). The 100% lability control is prepared in a matrix similar to the test products but utilizing a soluble zinc material (such as zinc sulfate) incorporated at an equivalent level on a zinc basis. The absorbance of the 100% lability control is measured as above for the test materials. The relative zinc lability is preferably greater than about 15%, more preferably greater than about 20%, and even more preferably greater than about 25%.

Using this methodology, the below examples demonstrate a material (basic zinc carbonate) that has intrinsically high lability in an anionic surfactant system compared to one (ZnO) with low intrinsic lability.

| | Relative Zinc Lability (%) | | |
|---|---|---|---|
| | In Water | In 6% sodium lauryl sulfate | Lability Benefit |
| Zinc Oxide | 86.3 | 1.5 | NO |
| Basic zinc | 100 | 37 | YES |
| carbonate | | | |

Zinc-containing layered structures are those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLM's) may have zinc incorporated in the layers and/or be components of the gallery ions.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLM's, which are often, but not always, synthetic, is layered doubly hydroxides, which are generally represented by the formula [M2+1−xM3+x(OH)2]x+Am−x/m.nH2O and some or all of the divalent ions (M2+) would be represented as zinc ions (Crepaldi, E L, Pava, P C, Tronto, J, Valim, J B J. Colloid Interfac. Sci. 2002, 248, 429-42).

Yet another class of ZLM's can be prepared called hydroxy double salts (Morioka, H., Tagaya, H., Karasu, M, Kadokawa, J, Chiba, K Inorg. Chem. 1999, 38, 4211-6). Hydroxy double salts can be represented by the general formula [M2+1−xM2+1+x(OH)3(1−y)]+ An−(1=3y)/n.nH2O where the two metal ion may be different; if they are the same and represented by zinc, the formula simplifies to [Zn1+x(OH)2]2x+2xA−.nH2O. This latter formula represents (where x=0.4) common materials such as zinc hydroxychloride and zinc hydroxynitrate. These are related to hydrozincite as well wherein a divalent anion replace the monovalent anion. These materials can also be formed in situ in a composition or in or during a production process.

These classes of ZLM's represent relatively common examples of the general category and are not intended to be limiting as to the broader scope of materials which fit this definition.

Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn5(OH)6(CO3)2 but the actual stoichiometric ratios can vary slightly and other impurities may be incorporated in the crystal lattice.

d) Polar Solvent Carrier

The composition of the present invention comprises a polar solvent carrier for delivering the essential components, and for providing suitable rheology with the hydrophilic clay material. The polar solvent also serves as solvent for incorporating water-soluble oral care actives and other components.

The polar solvent is comprised by weight of the entire composition at from about 1% to about 95%, preferably from about 30% to about 70%.

Polar solvents useful herein include water, polyhydric alcohols such as glycerin, 1,3-butylene glycol, propylene glycol, hexylene glycol, propane diol, ethylene glycol, diethylene glycol, dipropylene glycol, diglycerin, sorbitol, and other sugars which are in liquid form at ambient temperature. Also useful herein are water soluble alkoxylated nonionic polymers such as polyethylene glycol.

In one preferred embodiment, the present composition can comprise a relatively high level of water for providing a cost effective product. In such preferred embodiment, water is comprised at from about 30% to about 95%, more preferably from about 50% to about 70% of the entire composition.

Commercially available polar solvents herein include: glycerin available from Asahi Denka; propylene glycol with tradename LEXOL PG-865/855 available from Inolex, 1,2-PROPYLENE GLYCOL USP available from BASF; 1,3-butylene glycol available from Daisel Kagaku Kogyo; dipropylene glycol with the same tradename available from BASF; diglycerin with tradename DIGLYCEROL available from Solvay GmbH, polyethylene glycol with the tradename PEG 300 available from Doe Chemical Company, and sorbitol 70% solution available from Khalista (Liuzhou) Chemical Industries, Ltd.

Free of Abrasive Material

The composition of the present invention is substantially free of abrasive material. Namely, the composition of the present invention has no abrasive material that is intentionally included. It has been surprisingly found that, the present composition can deliver the benefits of a regular dentifrice composition, including those which are typically attributed to the use of abrasive material, such as cleaning, stain removal, plaque removal, calculus removal, and others. Absence of abrasive material also accommodates inclusion of certain active agents such as cetylpyridinium chloride, which otherwise have poor compatibility with abrasive materials such as silica, and therefore cannot be included in a dentifrice composition. Further, due to the absence of abrasive material, various aesthetics for the dentifrice product may be pursued. For example, a composition of transparent appearance may be made.

Abrasive materials from which the present composition is substantially free of, are defined as materials having a DOA value of no more than about 150 ml/100 g, preferably no more than about 100 ml/100 g, and having a particle size of from about 5 μm to about 50 μm. Abrasive materials herein can be distinguished herein from those materials useful as binders.

Abrasive materials from which the present composition is free of, include: inorganic minerals such as silica gels and precipitates; aluminas; hydrated alumina, calcium carbonate, titanium dioxide, talc, calcium dioxide, and resinous abrasive materials such as particulate condensation products of urea and formaldehyde.

Additional Binder Material

The composition of the present invention may further comprise an additional binder material which further builds rheology to the composition, or provides certain mouth feel upon usage. When included, the additional binder material is selected from those compatible with the hydrophilic clay material, and are included such that the total amount of hydrophilic clay material and additional binder material is from about 0.1% to about 10% of the entire composition, and provides a viscosity of from about 10,000 Pas to about 450,000 Pas, preferably from about 10,000 Pas to about 250,000 Pas.

Modified cellulose polymers are useful binder materials herein for building continuous structure to the composition. Modified cellulose polymers useful herein include sodium carboxymethyl cellulose, polyvinylpyrrolidone, hydroxyethylpropylcellulose, hydroxybutyl methyl cellulose, hydroxypropy methyl cellulose, and hydroxyethyl cellulose. Particularly useful commercially available materials include sodium carboxymethyl cellulose by the tradename BLANOSE series, OPTICEL 100, and AQUALON series from Hercules; hydroxyethylpropylcellulose by the tradename KLUCEL series from Hercules, hydroxypropy methyl cellulose by the tradename TF-E25 from YIXING CITY NO.8 CHEMICAL PLANT and by the tradename BENECEL series from Hercules.

Carboxyvinyl anionic polymers are useful binder materials herein for building a three dimentional structure in combination with the hydrophilic clay materials disclosed hereinabove. Carboxyvinyl anionic polymers useful herein include those by the tradename of CARBOPOL series (or CARBOMER) such as CARBOPOL 956, 934, 940, 941, 1342, ETD 2020, ULTREZ 10″ from Noveon Inc., CARBOPOL 934, 940, 974 P from B. F. Goodrich and SYNTHALEN K available from 3V Company.

Natural gum derived anionic polymers are useful binder materials herein for effectively adding viscosity. Natural gum derived anionic polymers useful herein include carrageenan, xanthan gum, gellan gum, and locust bean gum. Particularly useful commercially available materials include xanthan gum by the tradename KELDENT series available from CP Kelco, and by the tradename RHODICARE series from Rhodia; gellan gum by the tradename KELCOGEL series from CP Kelco; and locust bean gum by the tradename GELLOID LB series from FMC BioPolymer.

Also useful as binder material herein are starch and carrageenan. Carrageenan is commercially available by tradename VISCARI series and GELCARIN series from FMC BioPolymer, by the tradename MEYPRO-SOL series from Meyhall AG, and by the tradename GENUVISCO series, GENU series and GENUTINE series from CP Kelco.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such additional components generally are used individually at levels of no more than about 5% by weight of the composition.

Surfactants may be incorporated in the components of the present invention as an ingredient to aid in the thorough dispersion of the dentifrice throughout the oral cavity when applied thereto, as well as, to improve cosmetic acceptability and the foaming properties. The surfactants which can be included in the present composition include anionic, nonionic or amphoteric compounds, anionic compounds being preferred.

Suitable examples of anionic surfactants are higher alkyl sulfates such as potassium or sodium lauryl sulfate which is preferred, higher fatty acid monoglyceride monosulfates, such as the salt of the monosulfated monoglyceride of hydrogenated coconut oil fatty acids, alkyl sulfonates such as sodium dodecyl benzene sulfonate, higher fatty sulfoacetates, higher fatty acid esters of 1,2 dihydroxypropane sulfonate.

Examples of water soluble nonionic surfactants are condensation products of ethylene oxide with various hydrogen-containing compounds that are reactive therewith and have long hydrophobic chains (e.g., aliphatic chains of about 12 of 20 carbon atoms), which condensation products contain hydrophilic polyoxyethylene moieties, such as condensation products of poly (ethylene oxide) with fatty acids, fatty alcohols, fatty amides and other fatty moieties, and with propylene oxide and polypropylene oxides, e.g., Pluronic materials such as Pluronic F127.

The surfactant can be present in the present composition at a concentration of from about 0.5 to about 10.0% by weight, preferably about 1 to about 2% by weight.

Other ingredients which may be incorporated in the dentifrice composition of the present invention include pigments, dyes, flavoring and sweetening materials. For example, a striped product is obtained in accordance with the present invention wherein colorants of contrasting colors are incorporated in each of the components used in the practice of the present invention, the colorants being pharmacologically and physiologically nontoxic when used in the suggested amounts. Colorants used in the present invention include both pigments and dyes.

Pigments useful in the present invention include non-toxic, water insoluble inorganic pigments such as titanium dioxide and chromium oxide greens, ultramarine blues and pinks and ferric oxides as well as water insoluble dye lakes prepared by extending calcium or aluminum salts of FD&C dyes on alumina such as FD&C Green #1 lake, FD&C Blue #2 lake, FD&C R&D #30 lake, FD&C #5 Yellow and FD&C # Yellow 15 lake, The concentration of the dye in the present composition is no more than about 3% by weight. The pigments herein are distinguished from the abrasive materials, which the present composition is substantially free from, as pigments herein have a particle size in the range of from 100 to about 1000 microns, preferably from about 250 to about 500 microns. When present, the pigments are included in the present composition at from about 0.5% to about 3% by weight.

Any suitable flavoring or sweetening material may also be incorporated in the components of the present invention. Examples of suitable flavoring constituents are flavoring oils, e.g., oils of spearmint, peppermint, wintergreen, sassafras, clove, sage, eucalyptus, marjoram, cinnamon. Lemon, and orange, and methyl salicylate. Suitable sweetening agents include sucrose, lactose, maltose, sorbitol, xylitol, sodium cyclamate, perillartine, and sodium saccharin. Suitably, flavor and sweetening agents may together comprise from 0.01% to 5% by weight or more of the preparations.

Enzyme inhibitor, botanical extracts, natural herb extracts, and others may be included.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

The following dentifrice compositions are formed by the following components using the method of preparation described herein.

Compositions for Examples 1-5:

| Component | Ex. 1 | Ex. 1 | Ex. 3 | Ex. 4 | Ex. 5 |
|---|---|---|---|---|---|
| Sodium Fluoride *1 | 0.243 | 0.243 | 0.243 | 0.243 | 0.243 |
| Sorbitol (70% sol.) *2 | 39.299 | 39.299 | 25 | 20 | |
| Glycerin *3 | | | 5 | | 20 |
| Polyethylene Glycol *4 | | | | 10 | |
| Sodium Lauryl Sulfate | 5 | 7.5 | 1 | 7.5 | 10 |
| (28% sol.) *5 | | | | | |
| Sodium Carboxymethyl | 1.0 | 1.0 | 1 | | 2.0 |
| Cellulose *6 | | | | | |
| Carbopol *7 | 0.6 | 0.6 | 1.0 | | 1.0 |
| Xanthan Gum *8 | 0.35 | 0.35 | 0.5 | 0.35 | |
| Carrageenan *9 | | | 0.5 | | |
| Synthesized Layered | 1.0 | 1.0 | 0.4 | 0.5 | |
| Magnesium Silicate | | | | | |
| 0.2% Fluoride *10 | | | | | |
| Synthesized Layered | | | | | 0.2 |
| Magnesium Silicate *11 | | | | | |
| Amorphous Precipitated | | | 0.5 | | |
| Silica *12 | | | | | |
| Fumed Silica *13 | | | | | 0.5 |
| Mica, Titanium | | | | | 0.5 |
| Dioxide Coated *14 | | | | | |
| Sodium Hydroxide | 0.54 | 0.54 | 0.54 | 0.54 | |
| Solution (50%) *15 | | | | | |
| Tetrasodium | 0.05 | 0.05 | 0.05 | 0.05 | |
| Pyrophosphate *16 | | | | | |
| Sodium Phosphate, | | | | | 0.4 |
| Monobasic, | | | | | |
| Monohydrate *17 | | | | | |
| Tribasic Sodium | | | | | 0.9 |
| Phosphate, NF | | | | | |
| (Dodecahydrate) *18 | | | | | |
| Flavor | 0.80 | 0.80 | 0.50 | 0.80 | 1.00 |
| Sodium Phytate 20% | 5.0 | 2.5 | 25.0 | 2.5 | 1.25 |
| Solution *19 | | | | | |
| Sodium Saccharin *20 | 0.25 | 0.25 | 0.25 | 0.25 | 0.25 |
| Dyes | 0.0008 | 0.0008 | 0.0008 | 0.0008 | 0.0008 |

| | |
|---|---|
| Deionized Water | QS to 100% |

Compositions for Examples 6-10

| Component | Ex. 6 | Ex. 7 | Ex. 8 | Ex. 9 | Ex. 10 |
|---|---|---|---|---|---|
| Sodium Fluoride *1 | | | 0.243 | 0.243 | 0.243 |
| Stannous Fluoride *21 | 0.454 | 0.454 | | | |
| Sorbitol (70% sol.) *2 | 39.299 | 39.299 | 39.299 | 39.299 | 39.299 |
| Sodium Lauryl Sulfate | 1 | 3 | 5 | 7.5 | 10 |
| (28% sol.) *5 | | | | | |
| Sodium Carboxymethyl | 2.0 | 2.0 | 1.0 | 1.0 | 1.0 |
| Cellulose *6 | | | | | |
| Carbopol *7 | | | 0.6 | 0.6 | 0.6 |
| Xanthan Gum *8 | 0.35 | 0.35 | 0.35 | 0.35 | 0.35 |
| Synthesized Layered | 0.2 | 0.2 | 0.2 | 0.2 | 0.2 |
| Magnesium Silicate | | | | | |
| 0.2% Fluoride *10 | | | | | |
| Sodium Hydroxide | 0.54 | 0.54 | 0.54 | 0.54 | 0.54 |
| Solution (50%) *15 | | | | | |
| Tetrasodium | 0 | 2.0 | 5.0 | 5.0 | 10.0 |
| Pyrophosphate *16 | | | | | |
| Flavor | 0.80 | 0.80 | 0.75 | 0.75 | 0.75 |
| Triclosan *22 | | | 0.28 | | |
| Cetylpyridinium | | | 0.045 | | |
| Chloride *23 | | | | | |
| Zinc Carbonate *24 | | | | 1.0 | |
| Sodium Phytate 20% | 25.0 | 17.5 | 5.0 | 2.5 | 0 |
| Solution *19 | | | | | |
| Sodium Saccharin *20 | 0.25 | 0.25 | 0.25 | 0.25 | 0.25 |
| Dyes | 0.0008 | 0.0008 | 0.0008 | 0.0008 | 0.0008 |

| | |
|---|---|
| Water | QS to 100% |

Definition of Components * 1 Sodium Fluoride: NaF, available from Jinan Chemical Industry Co. Ltd. *2 Sorbitol (70% sol.): Sorbitol 70% solution, available from Khalista (Liuzhou) Chemicals Industries Ltd. *3 Glycerin: Glycerin, available from Asahi Denka *4 Polyethylene Glycol: PEG-300, available from Doe Chemical Company *5 Sodium Lauryl Sulfate (28% sol.): SLSS, available from Rhodia Specialty Chemical Wuxi Co. Ltd. *6 Sodium Carboxymethyl Cellulose: CMC, available from Zhangjiagang city Sanhui Chemical Industry Co., Ltd *7 Carbopol: Carbomer 956, available from Noveon, Inc. *8 Xanthan Gum: KELDENT, available from CP Kelco Inc. *9 Carrageenan: GELCARIN TP911 available from FMC Corporation food ingredients division *10 Synthesized Layered Magnesium Silicate 0.2% Fluoride: LAPONITE DF, available from Rockwood Additives Limited *11 Synthesized Layered Magnesium Silicate: LAPONITE D, available from Rockwood Additives Limited * 12 Amorphous Precipitated Silica: Zeodent 165, available from J. M. Huber Company *13 Fumed Silica: AEROSOL, available from Cabot & Degussa Corporation *14 Mica, Titanium Dioxide Coated: available from Rona *15 Sodium Hydroxide Solution (50%): NaOH 50%, available from Guangzhou Chemical Company. *16 Tetrasodium Pyrophosphate: Sodium Pyrophosphate, Tetra (anhydrous), available from Lianyungang Duoling Fine Chemical Co. Ltd. *17 Sodium Phosphate, Monobasic, Monohydrate: MSP, available from Jiangsu Chengxing Phosphate Chemical Co. Ltd *18 Tribasic Sodium Phosphate: TSP, available from Jiangsu Jiangyin Phosph Chemicals * 19 Sodium Phytate 20% Solution: available from Sichuan Chengdu Yason *20 Sodium Saccharin: available from Suzhou Fine Chemical *21 Stannous Fluoride: SnF2, available from Hashimoto *22 Triclosan: available from Ciba-Geigy Chemicals Ltd. *23 Cetylpyridinium Chloride: available from Cambrex company *24 Zinc Carbonate: available from Bruggemann Chemical
Method of Preparation

The dentifrice compositions of Examples 1-10 may be made by any method known to one skilled in the art, and are suitably prepared as follows.

First, the hydrophilic clay material (component numbers 10-13) is dissolved in part of water in a container at room temperature, using Propeller Mixer mix until homogeneous. In a separate main mix pot, sorbitol solution (component number 2), glycerin (component number 3), polyethylene glycol (component number 4), sodium hydroxide solution (component number 15), dye and pigment (component number 14) as present in the composition, and remaining part of water, are mixed together with an agitator at 25 to 35 rpm rotation speed at elevated temperature.

All remaining components except flavor and surfactant (component number 5) are added to main mix pot. The pot is hermetically closed, vacuum is built to around 100 mmhg, homogenizer is turned at a rotation rate of 2400 to 3500 rpm. The obtained mixture is further de-aerated.

To this is added the mixture made at first step. The obtained mixture is mixed and de-aerated. Finally, flavor and surfactant (component number 5) are added, the pot is again hermetically closed, mixed by homogenizer turned at a rotation rate of 2400 to 3500 rpm for final homogenization, and de-aerated.

The obtained product is pumped out of the container and delivered to a primary packaging such as laminate tube.

The Example compositions herein have many benefits suitable for a dentifrice product. All compositions provide lower abrasion to teeth surface, and are safer to teeth enamel compared to toothpastes containing abrasive material. All compositions provide soft mouth feel, and easier dispersion, and improved approach in the between-teeth area compared to toothpastes containing abrasive material. All compositions provide better consumer noticeability of flavor as compared to toothpastes containing abrasive material and the same level of the same flavor. Further, all compositions can be manufactured at an economical cost. The compositions of Examples 1-5 provide anti-caries, anti-plaque, whitening, and anti-calculus benefit. The compositions of Examples 6-7 provide anti-caries, anti-plaque, whitening, anti-calculus, and anti-gingivitis benefit. The compositions of Examples 8-9 provide anti-caries, anti-plaque, whitening, anti-calculus, and anti-microbial benefit. The composition of Example 10 provides anti-caries, anti-plaque, and anti-calculus benefit.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dentifrice composition comprising:
(a) a hydrophilic clay material;
(b) a phytic acid compound;
(c) an effective amount of an oral care active; and
(d) a polar solvent carrier;
wherein the composition is substantially free of abrasive material.

2. The dentifrice composition of claim 1 comprising:
(a) from about 0.1% to about 10% of the hydrophilic clay material; and
(b) from about 0.01% to about 20% of the phytic acid compound.

3. The dentifrice composition of claim 1 wherein the hydrophilic clay material is selected from the group consisting of a natural and synthesized layered silicate minerals, fumed silicas, thickening precipitated silica, and mixtures thereof.

4. The dentifrice composition of claim 3 wherein the hydrophilic clay material is a synthesized layered magnesium silicate.

5. The dentifrice composition of claim 1 further comprising an additional binder material selected from the group consisting of modified cellulose polymers, carboxyvinyl polymers, natural gum derived anionic polymers, and mixtures thereof.

6. The dentifrice composition of claim 1 wherein the oral care active is selected from the group consisting of anti-calculus agents, stannous ion sources, fluoride ion sources, whitening agents, anti-microbial agents, anti-plaque agents, anti-inflammatory agents, nutrients, antioxidants, anti-viral agents, analgesic and anesthetic agents, zinc-containing layered material, and mixtures thereof.

7. The dentifrice composition of claim 6 wherein the oral care active is selected from the group consisting of fluoride ion sources, stannous ion sources, pyrophosphate salts, polyphosphate salts, hydrogen peroxide, triclosan, and mixtures thereof.

8. The dentifrice composition of claim 6 wherein the oral care active is an anti-cavity active selected from the group consisting of cetylpyridinium chloride, stannous ion sources, and mixtures thereof.

9. The dentifrice composition of claim 6 wherein the oral care active is a zinc-containing layered material having a relative zinc lability of greater than about 25%.

10. The dentifrice composition of claim 9 wherein the zinc-containing layered material is selected from the group consisting of basic zinc carbonate, zinc carbonate hydroxide, zinc copper carbonate hydroxide, aurichalcite, copper zinc carbonate hydroxide, rosasite, phyllosilicate containing zinc ions, layered double hydroxide, hydroxyl double salts, and mixtures thereof.

11. The dentifrice composition of claim 9 wherein the zinc-containing layered material is basic zinc carbonate.

12. The dentifrice composition of claim 1 wherein the polar solvent carrier comprises, by weight of the entire composition, from about 30% to about 95% water.

13. A method of cleaning teeth absent the use of abrasive material comprising the step of brushing the teeth with the dentifrice composition of claim 1.
